(19) United States
(12) Patent Application Publication
(10) Pub. No.: US 2012/0245539 A1
(43) Pub. Date: Sep. 27, 2012

(54) LACRIMAL IMPLANTS INCLUDING SPLIT AND INSERTABLE DRUG CORE

(75) Inventors: Sascha K. Zarins, Saratoga, CA (US); David J. Scott, Redwood City, CA (US); David Tholfsen, San Leandro, CA (US)

(73) Assignee: QLT INC., VANCOUVER, BC (CA)

(21) Appl. No.: 13/512,277

(22) PCT Filed: Nov. 26, 2010
(86) PCT No.: PCT/US10/58129

§ 371 Date: Jun. 6, 2012

Related U.S. Application Data
(60) Provisional application No. 61/283,100, filed on Nov. 27, 2009.

(51) Int. Cl. A61F 9/00; B23P 17/00; A61M 31/00

(52) U.S. Cl. 604/294; 29/428

(57) ABSTRACT

Lacrimal implants providing secure, wedgable retention within a lacrimal punctum and associated canaliculus of an eye are disclosed. The lacrimal implants can comprise an implant body, including first and second portions, extending from a proximal end of the first portion to a distal end of the second portion. The second portion includes a retention projection. The implant body can also include a cavity longitudinally extending from the proximal end of the first portion toward the second portion, wherein the cavity is shaped and sized to receive an insertable actuator, and wherein the retention projection is configured to bias outward or change orientation when the insertable actuator is seated within the cavity.

### CROSS-REFERENCE TO RELATED APPLICATIONS

This application claims the benefit of priority from U.S. Provisional Patent Application Ser. No. 61/283,100, filed Nov. 27, 2009, the entire content of which is incorporated herein by reference.

### TECHNICAL FIELD

This patent document pertains generally to ophthalmic devices, and particularly to ocular implants. More particularly, but not by way of limitation, this patent document pertains to lacrimal implants, methods of making such implants, and methods of treating ocular, respiration, inner ear or other diseases or disorders (e.g., pulmonary or immunological disorders) using such implants.

### BACKGROUND

Dry eye, including keratoconjunctivitis sicca, is a common ocular condition that can require therapy. Dry eye has been experienced by a broad demographic band, and is common in elderly individuals. A variety of current treatment modalities target physiological conditions that contribute to dry eye, including augmentation of normal tear fluid, enhancement of tear film component production, and methods to enhance the residence time of tears, such as blocking the tear flow from an eye into and through a lacrimal canaliculus.

Many current tear flow blockage techniques have drawbacks, including being irreversible in nature. For instance, some tear flow blockage techniques involve closing a canalicular canal by stitching the associated punctal opening shut or by using electrical or laser cauterization to seal the punctal opening. Although such procedures can provide the desired result of blocking tear flow to treat dry eye, they are not reversible without reconstructive surgery.

In addition to dry eye symptom relief, a variety of challenges face patients and physicians in the area of ocular, respiration and inner ear disease or disorder management, including adequate drug or other therapeutic agent delivery to the eyes, nasal passage or inner ear. In ocular management, for example, many current ocular drug delivery systems require repetitive manual administration and are often ineffective due to a lack of patient compliance or inadequate drug concentrations reaching the eye. For instance, when an eye drop is instilled in an eye, it often overfills the conjunctival sac (i.e., the pocket between the eye and the lids) causing a substantial portion of the drop to be lost due to overflow of the lid margin and spillage onto the cheek. A large portion of the drop remaining on the ocular surface can be washed away into and through a lacrimal canaliculus shortly after application, thereby diluting the concentration of the drug before it can absorbingly treat the eye. Moreover, topically applied drugs often have a peak ocular effect for about two hours post-application, after which additional applications of the drugs should be, but are often not, administered to maintain the desired drug therapeutic benefit.

In a field different from ocular management, control of respiration-related (e.g., allergies) and inner ear diseases or disorders often requires repetitive manual digestion or other intake of a medication (e.g., drugs or other therapeutic agents), and can be ineffective due to a lack of patient compliance or non-localized drug delivery.

### SUMMARY

The present inventors have recognized various promising techniques to increase the residence time of tears on an eye and delivery of drug or other therapeutic agent to the eye, nasal passage, inner ear or other bodily system. These techniques can include placing a removable, and optionally drug releasing, lacrimal implant through a lacrimal punctum and into the associated canaliculus. It is believed that by designing lacrimal implants that utilize one or more features of the nasolacrimal drainage system (e.g., by mimicking the shape of the lacrimal canaliculus or lacrimal canaliculus ampulla), patient comfort and implant retention in the ocular anatomy can be satisfied. In this way, the present lacrimal implants can overcome some of the drawbacks associated with current dry eye relief, such as being irreversible in nature, and manual drop or digestion-based drug administration, such as poor patient compliance, waste, untimely application, or non-localized delivery.

Further yet, the present inventors have recognized that a lacrimal implant can benefit from one or more of: the ability to be easily implanted and removed without much biasing of the lacrimal punctum or associated canaliculus, the ability to be securely retainable in the lacrimal canaliculus upon implantation, optionally without being pre-sized to a particular lacrimal punctum or canaliculus diameter, the ability to permit tear fluid, drug or other agent to flow into the nasolacrimal system, and, when made and used as a drug delivery system, the ability to allow for the sustained, localized release of one or more drugs or other therapeutic agents at a desired therapeutic level for an extended period of time.

In light of these recognitions, lacrimal implants for treating diseases or disorders are disclosed. More particularly, lacrimal implants, methods of making such implants, and methods of treating ocular, respiration, inner ear, pulmonary or immunological diseases or disorders using such implants are disclosed.

An example of a lacrimal implant comprises an implant body, including first and second portions, extending from a proximal end of the first portion to a distal end of the second portion. The second portion includes a retention projection. The implant body can also include a cavity longitudinally extending from the proximal end of the first portion toward the second portion. The cavity is shaped and sized to receive an insertable actuator, and the retention projection is configured to bias outward or change orientation when the insertable actuator is seated within the cavity.

An example of a method of forming a lacrimal implant includes forming an implant body (including first and second portions of the implant body), extending the implant body from a proximal end of the first portion to a distal end of the second portion, forming a cavity longitudinally extending from the proximal end of the first portion toward the second portion, and shaping and sizing the cavity to receive an insertable actuator, and forming a retention projection in the second portion to bias outward or change orientation when the insertable actuator is seated within the cavity.

These and other embodiments, advantages, and aspects of the present lacrimal implants and methods will be set forth in part in following Detailed Description. This Exemplary Embodiment section is intended to provide an overview of subject matter of the present patent application. It is not intended to provide an exclusive or exhaustive explanation of the present inventive implants. The Detailed Description is included to provide further information about the present patent document.

### BRIEF DESCRIPTION OF THE DRAWINGS

In the drawings, like numerals can be used to describe similar components throughout the several views. Like numerals having different letter suffixes can be used to represent different instances of similar components. The drawings illustrate generally, by way of example, but not by way of limitation, various embodiments discussed in the present document.

FIGS. 1-2 illustrate example views of anatomical tissue structures associated with the eye, certain of these tissue structures providing a suitable environment in which a lacrimal implant can be used.

FIGS. 3A-C illustrate views of an example lacrimal implant that is at least partially insertable through a lacrimal punctum and into the associated canaliculus, the lacrimal implant having a cut or void.

FIG. 4 illustrates a cross-sectional view of an example lacrimal implant, having a cut or void, that receives an insertable actuator.

FIGS. 5A-C illustrate views of another example of a lacrimal implant having a cut or void.

FIGS. 6A-C illustrate views of another example of a lacrimal implant having a cut or void.

FIG. 7A illustrates still another example of a lacrimal implant having a cut or void.

FIG. 7B illustrates the example lacrimal implant of FIG. 7A when a retention projection has a compressed orientation.

FIG. 8 is a flow diagram of an example method of manufacturing a lacrimal implant insertable through a lacrimal punctum and into the associated canaliculus.

FIGS. 9A-C illustrate views of an example insertion tool, which can be used to insert a lacrimal implant.

FIGS. 10A-E illustrate views of another example of a lacrimal implant having a cut or void.

FIGS. 11A-C illustrate views of an example insertion tool, which can be used to insert a lacrimal implant.

### DETAILED DESCRIPTION

In this patent document, lacrimal implants and related methods providing secure, wedgable retention within a lacrimal punctum and associated canaliculus of an eye are described. The lacrimal implants can comprise an implant body, including first and second portions, extending from a proximal end of the first portion to a distal end of the second portion. The second portion includes a retention projection. The implant body can also include a cavity longitudinally extending from the proximal end of the first portion toward the second portion, wherein the cavity is shaped and sized to receive an insertable actuator, and wherein the retention projection is configured to bias outward or change orientation when the insertable actuator is seated within the cavity.

In various examples, the lacrimal implant can further comprise a distinct drug insert or integrated drug or other agent disposed in at least one of the first portion or the second portion of the implant body, providing a sustained release of a drug or other therapeutic agent to one or more of an eye, nasal passage or inner ear system.

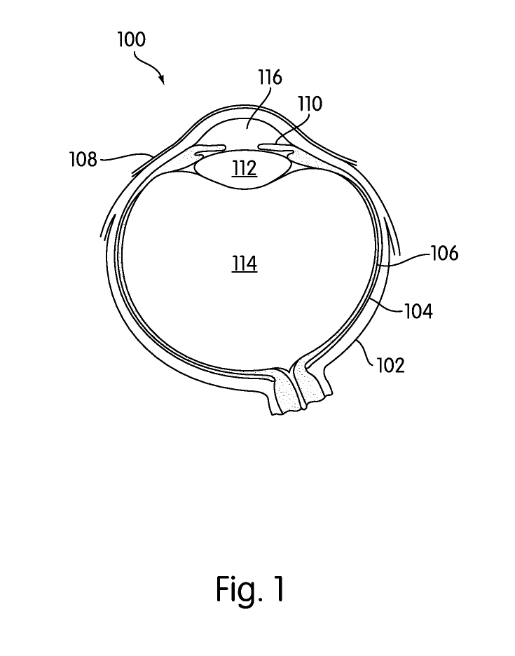

FIGS. 1-2 illustrate example views of anatomical tissue structures associated with an eye 100. Certain of the anatomical tissue structures shown can be suitable for treatment using the various lacrimal implants and methods discussed herein. The eye 100 is a spherical structure including a wall having three layers: an outer sclera 102, a middle choroid layer 104 and an inner retina 106. The sclera 102 includes a tough fibrous coating that protects the inner layers. It is mostly white except for the transparent area at the front, commonly known as the cornea 108, which allows light to enter the eye 100.

The choroid layer 104, situated inside the sclera 102, contains many blood vessels and is modified at the front of the eye 100 as a pigmented iris 110. A biconvex lens 112 is situated just behind the pupil. A chamber 114 behind the lens 112 is filled with vitreous humour, a gelatinous substance. Anterior and posterior chambers 116 are situated between the cornea 108 and iris 110, respectively and filled with aqueous humour. At the back of the eye 100 is the light-detecting retina 106.

The cornea 108 is an optically transparent tissue that conveys images to the back of the eye 100. It includes avascular tissue to which nutrients and oxygen are supplied via bathing with lacrimal fluid and aqueous humour as well as from blood vessels that line the junction between the cornea 108 and sclera 102. The cornea 108 includes a pathway for the permeation of drugs into the eye 100.

Turning to FIG. 2, other anatomical tissue structures associated with the eye 100 including the lacrimal drainage system, which includes a secretory system 230, a distributive system and an excretory system, are shown. The secretory system 230 comprises secretors that are stimulated by blinking and temperature change due to tear evaporation and reflex secretors that have an efferent parasympathetic nerve supply and secrete tears in response to physical or emotional stimulation. The distributive system includes the eyelids 202 and the tear meniscus around the lid edges of an open eye, which spread tears over the ocular surface by blinking, thus reducing dry areas from developing.

The excretory part of the lacrimal drainage system includes, in order of flow drainage, the lacrimal puncta, the lacrimal canaliculi, the lacrimal sac 204 and the lacrimal duct 206. From the lacrimal duct 206, tears and other flowable materials drain into a passage of the nasolacrimal system. The lacrimal canaliculi include an upper (superior) lacrimal canaliculus 208 and a lower (inferior) lacrimal canaliculus 210, which respectively terminate in an upper 212 and lower 214 lacrimal punctum. The upper 212 and lower 214 punctum are slightly elevated at the medial end of a lid margin at the junction 216 of the ciliary and lacrimal portions near a conjunctival sac 218. The upper 212 and lower 214 punctum are generally round or slightly ovoid openings surrounded by a connective ring of tissue. Each of puncta 212, 214 leads into a vertical portion 220, 222 of their respective canaliculus before turning more horizontal at a canaliculus curvature 250 to join one another at the entrance of the lacrimal sac 204. The canaliculi 208, 210 are generally tubular in shape and lined by stratified squamous epithelium surrounded by elastic tissue, which permits them to be dilated. As shown, a lacrimal canaliculus ampulla 252 exists near an outer edge of each canaliculus curvature 250.

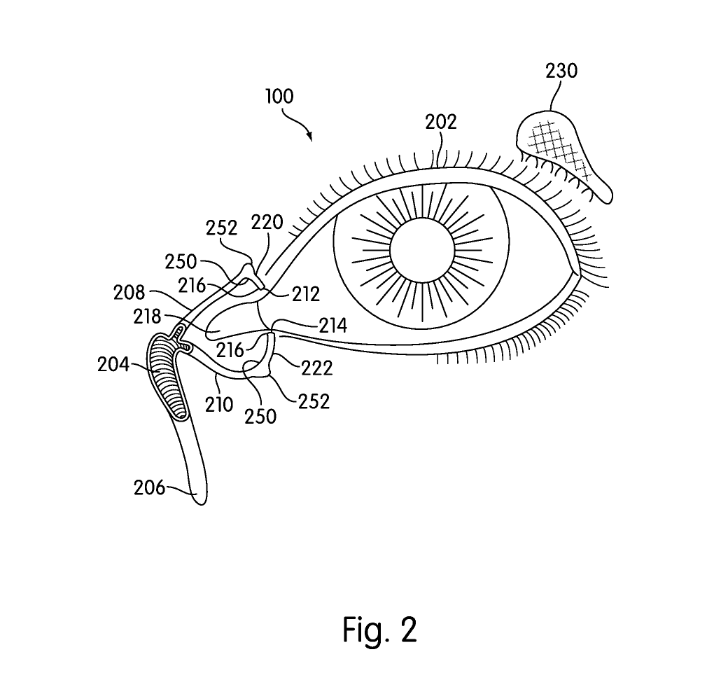

FIGS. 3A-C illustrate views of an example lacrimal implant 300 that can be insertable through a lacrimal punctum 212, 214 and into the associated canaliculus 208, 210 (FIG. 2). The insertion of the lacrimal implant 300 through the lacrimal punctum 212, 214 and into the associated canaliculus 208, 210 can allow for one or more of: inhibition or blockage of tear flow therethrough (e.g., to treat dry eyes) or the sustained delivery of a drug or other therapeutic agent to an eye (e.g., to treat an infection, inflammation, glaucoma or other ocular disease or disorder), a nasal passage (e.g., to treat a sinus or allergy disorder) or an inner ear system (e.g., to treat dizziness or a migraine).

As shown in this example, the lacrimal implant 300 can include an implant body 302, including a first portion 304 and a second portion 306. The implant body 302 extends from a proximal end 308 of the first portion 304 to a distal end 310 of the second portion 306. The second portion 306 includes a retention projection 312. The retention projection 312 can be instrumental in retaining or anchoring the lacrimal implant 300 in a lacrimal canaliculus 208, 210.

The implant body 302 also includes a cavity 314 longitudinally extending from the proximal end 308 of the first portion 304 toward the second portion 306. In certain examples, the cavity 314 is mostly cylindrical in shape. As shown in FIG. 3B, the cavity 314 longitudinally extends into, but not through, the second body portion 306, such that a distal end of the cavity 324 resides in the retention projection 312.

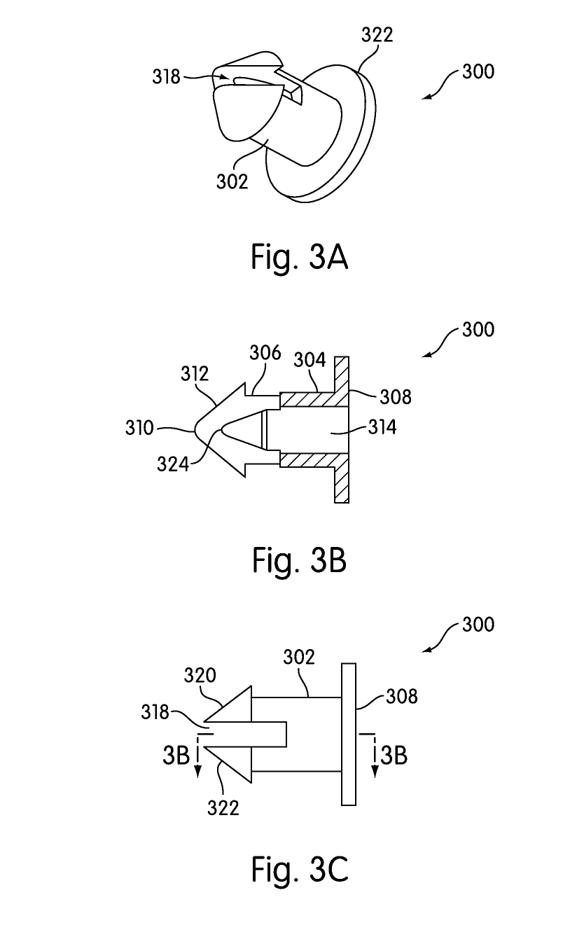

FIG. 4 shows a cross-sectional view of another example of a lacrimal implant 400. In this example, the cavity 414 is shaped and sized to receive an insertable actuator 416. In certain examples, the insertable actuator 416 has a cylindrical shape and size to mostly match a cylindrical diameter of the cavity 414. The insertable actuator 416 may alternatively have other shapes. For example, the insertable actuator 416 may have a plate shape having a width to mostly match a cavity width. The insertable actuator 416 may be retained by an interference fit. In certain examples, there is a clearance of about 0.5/1000 inches clearance on each side of the opening of the cavity 414 and an outer surface of the insertable actuator.

The retention projection 412 is configured to bias outward or change orientation when the insertable actuator 416 is seated within the cavity 414, post-canalicular insertion of the lacrimal implant 400. This can allow for better placement of the lacrimal insert than implants that are extended longitudinally, using an insert, for placement and then are allowed to expand when the insert is removed. Also, placement of implants that are extended longitudinally, using an insert, prior to placement may pierce a subject's canalicular anatomy due to the required longitudinal expansion.

In some examples, the diameter or width of the cavity 414 at the distal end of the implant 400 is less than the diameter or width of the cavity at the proximal end. The smaller diameter or width of the distal end of the cavity 414 and the diameter or width of the insertable actuator 416 causes the retention projection 412 to be biased in a lateral direction, relative to the cavity, when the insertable actuator 416 is fully seated in the cavity 414. Note that in these examples, the insertable actuator 416 does not have to expand (e.g., by fluid absorption) to cause the outward biasing of the retention projection 412. The outward biasing is caused by the difference in diameter or width of the insertable actuator 416 and the diameter or width of the cavity 414. In certain examples, the cavity 414 begins to taper from a wider diameter to a narrower diameter at a point proximal to the retention projection 412. In certain examples, the cavity 414 begins to taper from a wider diameter to a narrower diameter at a point within the retention projection 412.

Returning to FIGS. 3A-C, the lacrimal implant 300 includes a cut or void 318 extending from at least a portion of the cavity 314 through the retention projection 312 and substantially transverse to the longitudinal extension of the cavity 314. The cut or void 318 may be injection molded or may be cut into the lacrimal implant 300. In varying examples, the width of the cut or void 318 is less than the width of the insertable actuator. The cut or void 318 can define a first portion 320 of the retention projection 312 and a second portion 322 of the retention projection 312. The cut or void 318 allows the distal end of the lacrimal implant 300 to compress for insertion into punctal and canalicular anatomies of varying size. The retention projection 312 then expands outward after insertion of an insertable actuator to anchor the lacrimal implant 300. A larger cut or void 318 can allow for more compression of the lacrimal implant during insertion. However, a larger cut or void 318 may result in less outward expansion of the lacrimal implant 300. Thus, there is trade-off in sizing the cut or void 318. In certain examples, the width of the cut or void 318 is in a range of about 0.008 inches to about 0.012 inches. In certain examples, the width of the cut or void 318 is about 0.010 inches.

As shown in FIG. 4, a first portion 420 of the retention projection 412 is biased in a first outward direction and a second portion 422 of the retention projection 412 is biased in a second outward direction when the insertable actuator 416 is seated in the cavity 414. The cavity 414 extends at least partially through the cut or void to receive the insertable actuator 416 at least partially into the cut or void. The insertable actuator 416 may include an additional ring of material or glue wrapped around its distal end to increase the expansion of the retention projection 412 when the insertable actuator 416 is seated.

In some examples, the insertable actuator 416 may expand to cause or enhance the outward biasing. The insertable actuator 416 may include a material (e.g., Nitinol) having shape memory properties that causes the implant to expand after it is inserted in a punctum, such as by having a first shape at a relatively lower temperature and an expanded shape when exposed to warmer temperatures of the punctum. Examples of lacrimal implants including retention elements having a shape memory property can be found in commonly-owned De Juan et al., U.S. Patent Publication No. US-2007-0243230, entitled “NASOLACRIMAL DRAINAGE SYSTEM IMPLANTS FOR DRUG THERAPY,” filed Apr. 2, 2007, which is herein incorporated by reference in its entirety, including its description of retention elements.

In some examples, the insertable actuator 416 includes a hydrogel to expand through fluid absorption. Examples of lacrimal implants having hydrogel included in a retention element can be found in commonly-owned Jain et al., U.S. Patent Publication No. US-2009-0264861, entitled “LACRIMAL IMPLANTS AND RELATED METHODS,” filed Feb. 17, 2009, which is herein incorporated by reference in its entirety, including its description of retention elements. In some examples, the insertable actuator 416 expands in a cavity of changing diameter or width, and in some examples, the insertable actuator expands in a cavity of uniform diameter or width.

The portions or wings 420, 422 of the retention projection 412 may fold back along the lacrimal implant 400 during insertion. The lacrimal implant 400 may include a structure to strengthen the wings of the retention projection 412. The structure may include strengthening beams made of plastic or a shape memory metal (e.g., Nitinol) placed in each of the portions 420, 422 of the retention projection 412.

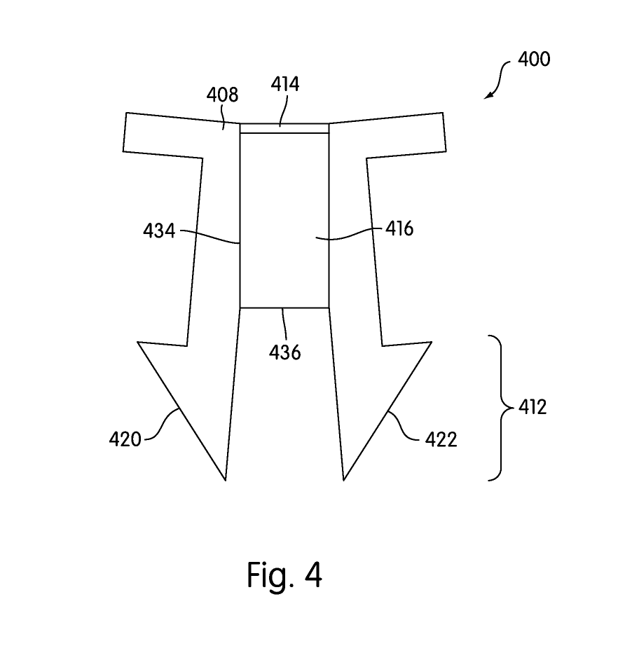

In some examples, the cut or void 318 of FIG. 3 does not extend completely through the retention projection 312. FIGS. 5A-C illustrate views of another example of a lacrimal implant 500 at least partially insertable into a lacrimal punctum. In the example shown, the cut or void 518 only extends partially through the retention projection 512. As discussed above, the cut or void 518 can define a first portion 520 of the retention projection 512 and a second portion 522 of the retention projection 512, and seating of an insertable actuator (not shown) can bias the first portion 520 in a first outward direction and bias the second portion in a second outward direction. Not having the cut or void 518 extend through the implant body inhibits the portions of the retention projection 512 from folding back during insertion. Also, retaining a tip at the distal end of the lacrimal implant 500 provides a tip to dilate tissue as the implant is inserted (i.e., provides a solid lead in), as well helping to inhibit distal dislodgement of the insertable actuator.

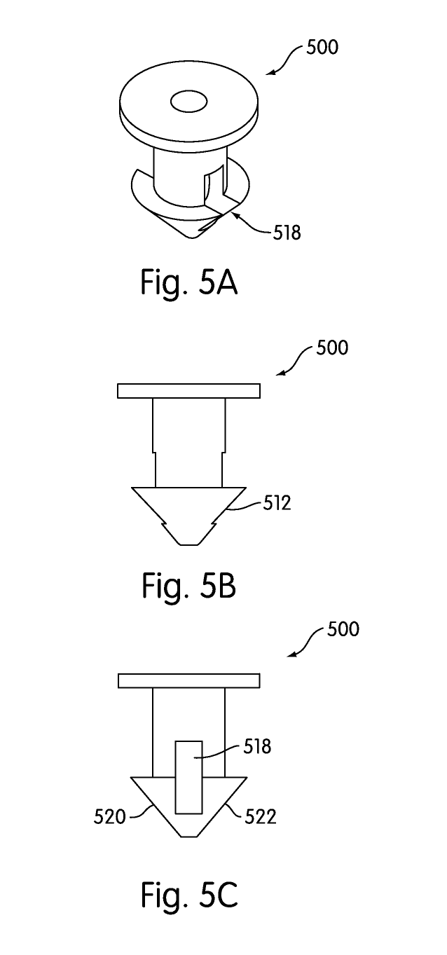

FIG. 6A shows another view of a lacrimal implant 600 having a cut or void 618 only partially extending through a retention projection 612. The lacrimal implant 600 is shown in its molded state without an actuator inserted. FIG. 6B shows the lacrimal implant 600 in a compressed state when being inserted. FIG. 6C shows the lacrimal implant 600 in an expanded state when an insertable actuator 616 or core is seated in the cavity.

Returning to FIG. 3, in some examples, the proximal end 308 of the first portion 304 of the implant body 302 includes a graspable projection 332 laterally protruding around at least a portion of its circumference. The projection can be graspable by a tweezers or forceps. In certain examples, the perimeter of the graspable projection 332 is numerically about equal to a perimeter of the proximal end of the retainment projection 312.

In some examples, the graspable projection 332 can be configured to seat against or near the punctal opening 212, 214, such as for inhibiting or preventing the lacrimal implant 300 from passing completely within the lacrimal canaliculus 208, 210, particularly when an actuator is being inserted. The graspable projection 332 also provides tactile or visual feedback information to an implanting user, e.g., as to whether the implant is fully implanted. In certain examples, the graspable projection 332 includes an inward-extending retaining lip that overhangs the cavity to proximally retain the insertable actuator.

The graspable projection 332 can extend laterally in a direction parallel to or away from an eye 100 when implanted. It is believed that the lateral protrusion of the graspable projection 332 may reduce irritation to the eye 100, as compared to a case in which a portion of the projection extends toward the eye 100.

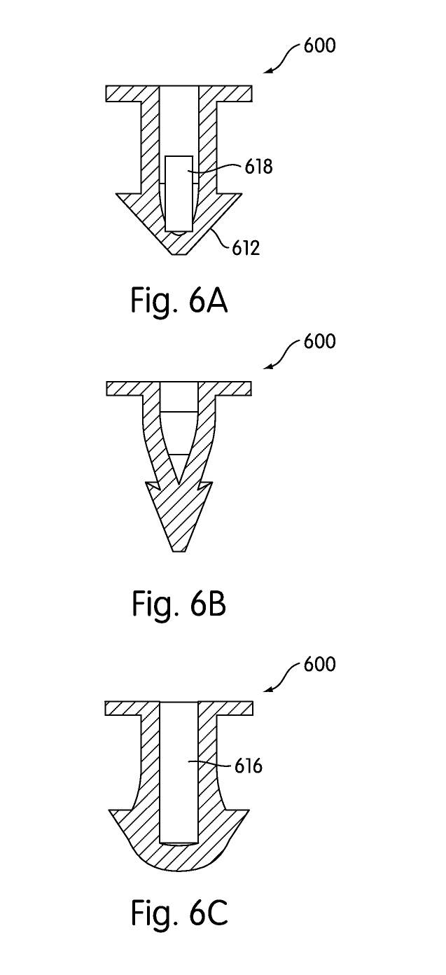

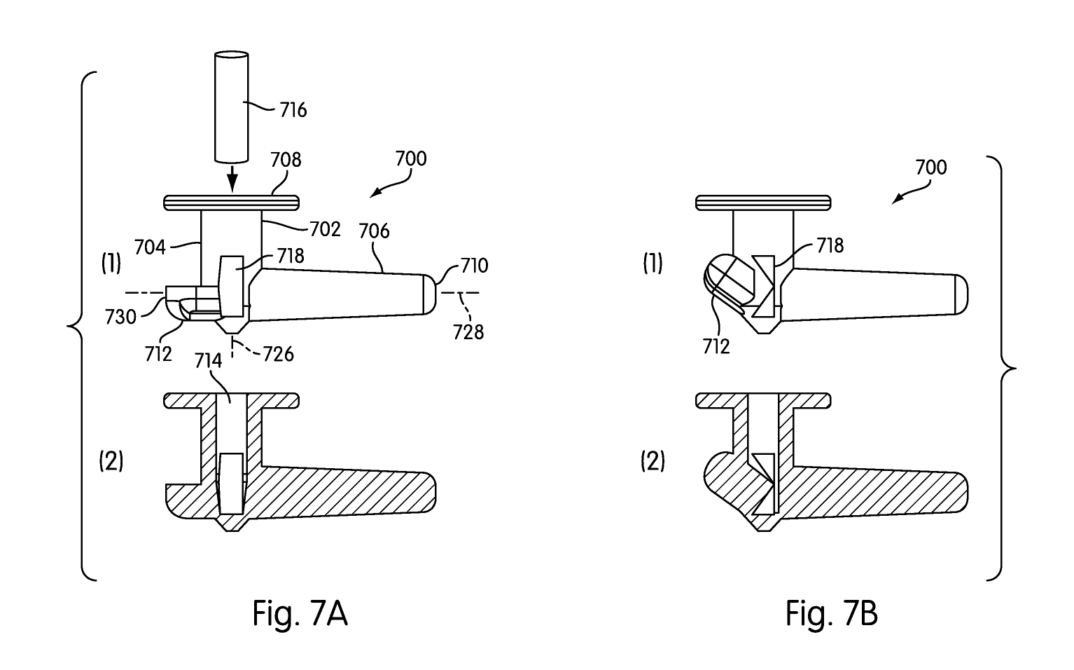

FIG. 7A illustrates another example of a lacrimal implant 700 at least partially insertable into a lacrimal punctum. As shown in this example, the lacrimal implant 700 can also include an implant body 702, including a first portion 704 and a second portion 706. The proximal end 708 of the first portion 704 of the implant body 702 defines a longitudinal proximal axis 726 and the distal end 710 of the second portion 704 of the implant body 702 defines a longitudinal distal axis 728. A non-linear angled intersection exists between the proximal axis 726 and the distal axis 728 for biasing at least a portion of the implant body 702 against at least a portion of the lacrimal canaliculus located at, or located more distal, to a canalicular curvature (250 in FIG. 2). In some examples, the angle of the intersection is between about 45 degrees and about 135 degrees. In various examples, the first portion distal end portion is integral with the second portion proximal end portion.

In some examples, the second portion 706 can include a length having a magnitude less than four times a length of the first portion 704. In one example, the second portion 706 can include a length of less than about 10 millimeters. Optionally, one or more portions of the implant body 702 can include an ovoid cross-sectional shape for anatomical fitting purposes.

A retention projection 712 is located at the proximal end 730 of the second portion 706. A cavity 714 extends longitudinally from the proximal end 708 of the first portion 704 toward the second portion 706. The cavity 714 is shaped and sized to receive an insertable actuator 716. The actuator shown has a cylindrical shape, but the insertable actuator 716 may have a different shape, as can the cavity 714. The proximal end 730 of the second portion 706 is biased toward a lacrimal canaliculus ampulla (252 in FIG. 2) when the insertable actuator 716 is seated in the cavity 714. In some examples, the lacrimal implant 700 includes a cut or void 718 in the first portion 704.

FIG. 7B shows that the cut or void 718 allows for compression of the retention projection 712 towards the first portion 704 during insertion. A sidewall of the cavity 714 is biased inward and the retention projection 712 is biased inward toward the first portion 704 when the insertable actuator 716 is not within the cavity 714. In some examples, the inward biasing is created when the lacrimal implant 700 is molded or otherwise formed.

After insertion, the retention projection 712 returns towards the position shown in FIG. 7A. Seating the insertable actuator 716 in the cavity biases the retention projection 712 outward from the first portion 704 orientation and towards the original position to anchor the lacrimal implant (e.g., plug) 700. In this example, the angle of intersection between the first portion 704 and the second portion 706 is about 90 degrees when the insertable actuator 716 is seated within the cavity. In certain examples, the left side of the cavity in the illustrations of FIGS. 7A and 7B may include a taper to narrow the cavity diameter or width near the distal end of the cavity (near the intersection of the first portion 704 and the second portion 706 of the implant body). This taper of the left side of the cavity 714 may further bias the retention projection outward from the first portion 704 orientation when the insertable actuator 716 is seated.

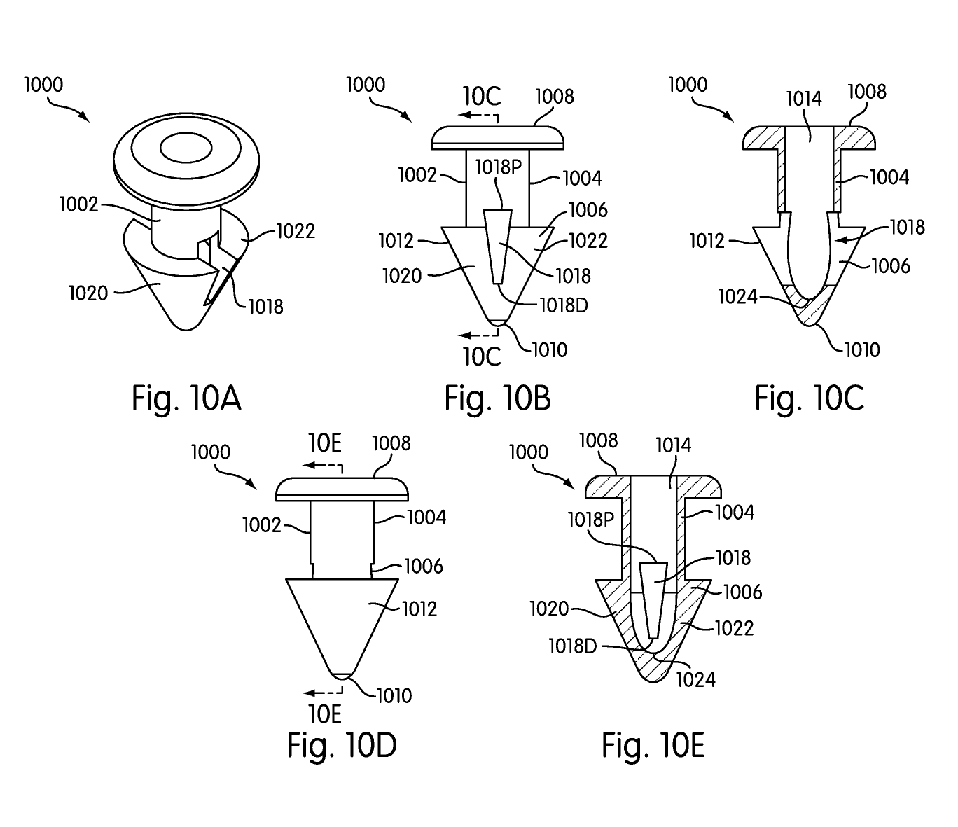

FIGS. 10A-E illustrate views of an example lacrimal implant 1000 that can be insertable through a lacrimal punctum 212, 214 and into the associated canaliculus 208, 210 (FIG. 2). The insertion of the lacrimal implant 1000 through the lacrimal punctum 212, 214 and into the associated canaliculus 208, 210 can allow for one or more of: inhibition or blockage of tear flow therethrough (e.g., to treat dry eyes) or the sustained delivery of a drug or other therapeutic agent to an eye (e.g., to treat an infection, inflammation, glaucoma or other ocular disease or disorder), a nasal passage (e.g., to treat a sinus or allergy disorder) or an inner ear system (e.g., to treat dizziness or a migraine).

As shown in this example, the lacrimal implant 1000 can include an implant body 1002, including a first portion 1004 and a second portion 1006. The implant body 1002 extends from a proximal end 1008 of the first portion 1004 to a distal end 1010 of the second portion 1006. The second portion 1006 includes a retention projection 1012. The retention projection 1012 can be instrumental in retaining or anchoring the lacrimal implant 1000 in a lacrimal canaliculus 208, 210.

The implant body 1002 also includes a cavity 1014 longitudinally extending from the proximal end 1008 of the first portion 1004 toward the second portion 1006. In certain examples, the cavity 1014 is mostly cylindrical in shape. As shown in FIG. 10B, the cavity 1014 longitudinally extends into, but not through, the second body portion 1006, such that a distal end of the cavity 1024 resides in the retention projection 1012.

As illustrated FIGS. 10A-C, the lacrimal implant 1000 includes a cut or void 1018 extending from at least a portion of the cavity 1014 through the retention projection 1012 and substantially transverse to the longitudinal extension of the cavity 1014. The cut or void 1018 may be injection molded or may be cut into the lacrimal implant 1000. In varying examples, the width of the cut or void 1018 is less than the width of the insertable actuator. The cut or void 1018 can define a first portion 1020 of the retention projection 1012 and a second portion 1022 of the retention projection 1012. The cut or void 1018 allows the distal end of the lacrimal implant 1000 to compress for insertion into punctal and canalicular anatomies of varying size. The retention projection 1012 then expands outward after insertion of an insertable actuator to anchor the lacrimal implant 1000. A larger cut or void 1018 can allow for more compression of the lacrimal implant during insertion. However, a larger cut or void 1018 may result in less outward expansion of the lacrimal implant 1000. Thus, there is trade-off in sizing the cut or void 1018. In certain examples, the width of the cut or void 1018 is about 0.010 inches at a proximal end 1018P and tapers to a width of about 0.003 inches at a distal end 1018D. In certain examples, the taper of the retention projection 1012 may be defined by an angle relative to the longitudinal axis of the lacrimal implant 1000 that is greater or less than the angle depicted in FIGS. 10A-E For example, in the example of the lacrimal implant 1000 illustrated in FIGS. 10A-E, the angle that defines the taper of the retention projection 1012 is smaller than the angle the defines the taper of the retention projection 312 of the lacrimal implant 300 illustrated in FIGS. 3A-C, which gives the retention projection 1012 a surface that is steeper or more pointed than the surface of the retention projection 312. In an example, the angle between the tapered surface of the retention projection 1012 and the longitudinal axis of the lacrimal implant 1000 may be about 26 degrees.

According to some examples, the lacrimal implant (e.g., lacrimal implant 400 of FIG. 4 or lacrimal implant 700 of FIG. 7) includes one or more therapeutic agents. In certain examples, the therapeutic agent(s) is included in the insertable actuator 416 or 716, thereby forming a drug core for the lacrimal implant 400 or 700. The agent-releasing drug core provides a sustained therapeutic agent (e.g., drug) release to an eye. In some examples, the insertable actuator/drug core is removable from the lacrimal implant 400 or 700 and a new insertable actuator/drug core may be inserted into the implant cavity 414 or 714 to provide a fresh supply of therapeutic agent.

In various examples, a drug core can include at least 21 micrograms, at least 42 micrograms, at least 44 micrograms, at least 66 micrograms, at least 81 micrograms, or at least 95 micrograms of a drug (e.g., latanoprost), such as is further discussed in commonly-owned Butuner et al., U.S. Patent Publication No. US-2009-0264861, entitled “SUSTAINED RELEASE DELIVERY OF ACTIVE AGENTS TO TREAT GLAUCOMA AND OCULAR HYPERTENSION,” filed May 8, 2009, and commonly-owned Utkhede, U.S. Patent Application No. 61/277,000, entitled “IMPROVED DRUG CORES FOR SUSTAINTED OCULAR RELEASE OF THERAPEUTIC AGENTS,” filed Sep. 18, 2009, both of which are incorporated by reference in their entirety, including their descriptions of drug or other agent concentration and excipient concentration.

In various examples, as shown in FIG. 4, the drug core can include a sheath body 434 disposed over at least a portion of the drug core of the insertable actuator 416 to define at least one core exposed surface. In certain examples, the drug core has a cylindrical shape and may have a diameter of about 0.022 inches and a length of about 0.072 inches. In certain examples, the sheath body 434 includes a polyimide tube around the drug core. The layer of polyimide may help prevent drug from being squeezed out of the drug core from pressure from the walls of the cavity 414.

An exposed surface of the drug core of the insertable actuator 416 can be located at or near the proximal end 408 of the implant body, for example, thereby allowing direct contact with a tear or a tear film fluid and release of a drug or other therapeutic agent from the drug core over a sustained time period when the lacrimal implant 400 is inserted through the lacrimal punctum 212, 214 and into the associated canaliculus 208, 210. The exposed surface can be flush or slightly below the proximal end 408 of the lacrimal implant 400 such that the drug core of the insertable actuator 416 does not protrude outside of the implant body. In some examples, the exposed surface of the drug core can be positioned above the proximal end 408 such that the drug core at least partially protrudes outside of the implant body.

In some examples, a barrier is included in distal end 436 of the drug core to prevent drug from being released into the nasolacrimal system. In certain examples, the barrier includes glue applied to the distal end 436 of the drug core of the insertable actuator. In some embodiments no barrier is present at the distal end 436 of the drug core and the drug core includes an agent to be released into the nasolacrimal system.

In some examples, the drug or other therapeutic agent release can occur, at least in part, via an exposed, non-sheath covered, surface of the insertable actuator 416. By controlling geometry of the exposed surface, a predetermined drug or agent release rate can be achieved. For instance, the exposed surface can be constructed with a specific geometry or other technique appropriate to control the release rate of the drug or other therapeutic agent onto an eye 100, such as on an acute basis or on a chronic basis, between outpatient doctor visits. Further description regarding effective release rates of one or more drugs or other therapeutic agents from a drug core or insert can be found in the afore-mentioned De Juan et al., “NASOLACRIMAL DRAINAGE SYSTEM IMPLANTS FOR DRUG THERAPY,” including its description of obtaining particular release rates.

In some examples, the drug core of the insertable actuator 416 can include a plurality of therapeutic agent inclusions, which can be distributed in a matrix. In some examples, the inclusions can comprise a concentrated (e.g., crystalline) form of the therapeutic agent. In some examples, the matrix can comprise a silicone matrix or the like, and the distribution of inclusions within the matrix can be substantially homogenous or non-homogeneous. In some examples, the agent inclusions can include droplets of oil, such as Latanoprost oil. In still other examples, the agent inclusions can comprise solid particles, such as Bimatoprost particles in crystalline form. In some examples, the drug core comprises a urethane-based (e.g., polyurethane) polymer or copolymer comprising therapeutic agent inclusions deliverable into the eye or surrounding tissues. The inclusions can be of many sizes and shapes. For instance, the inclusions can include microparticles having dimensions on the order of about lmicrometer to about 100 micrometers. Further discussion of drug-releasing or other agent-releasing drug inserts can be found in commonly-owned Utkhede et al., U.S. Patent Publication No. US-2009-0104243, entitled “DRUG CORES FOR SUSTAINED RELEASE OF THERAPEUTIC AGENTS,” filed Sep. 5, 2008, which is herein incorporated by reference in its entirety.

A second drug-releasing or other agent-releasing insert (e.g., a second drug core) may be disposed in the cavity 414 to provide a sustained drug or other therapeutic agent release to a nasal passage or inner ear system, for example. Thus, the first drug core may include a sustained release ocular agent, and the second drug core may include an agent deliverable, on a sustained release basis, to tissue of a nasolacrimal system.

The second drug core may be positioned distal to the first drug core in the cavity 414 of the lacrimal implant 400, or the second drug core may be located in the implant body second portion of the lacrimal implant of FIGS. 7A, 7B. An implant body septum may be positioned between the first drug core and second drug core to inhibit or prevent communication of a material (e.g., agent) between the two therapeutic agents.

Returning to FIGS. 3A-C, in various examples, the implant body 302 can be molded using an elastic material, such as silicone, polyurethane or other urethane-based polymer or copolymer, NuSil (e.g., NuSil 4840 with 2% 6-4800) or an acrylic of a non-biodegradable, partially biodegradable or biodegradable nature (i.e., erodeable within the body). Silicone, for example, is believed to be soft enough to be comfortable for patients and stiff enough to facilitate insertion by a caregiver physician. In some examples, polymers having a durometer of 40 and 48 are used to form the implant body 302.

In certain examples, the implant body 302 comprises an inert, non-expanding material, and in some examples, one or more therapeutic agents are integrated with the implant body 302. The implant body 302 can be configured to receive one or more drugs or other agents integrated throughout one or more body portions. In this way, the entire implant body 302, or portions thereof, can act as the drug-releasing or other agent-releasing insert. In certain examples, a first therapeutic agent is included in the insertable actuator 314 and a second therapeutic agent is included with the implant body 302.

In certain examples, release of an agent can be directed using a preformed opening(s) in an impermeable or substantially impermeable cover (e.g., parylene cover) surrounding portions of the implant body 302. In other examples, a permeable cover material can be used to allow for drug or other agent release.

According to some examples, the lacrimal implant 300 is designed for easier detection by a caregiver. For instance, a biocompatible colorant (e.g., green colorant) can be mixed with the elastic material of the implant body 302 allowing patients and their caregivers to more easily detect the implant and verify it remains in an implanted position. In some examples, the biocompatible colorant can be mixed with materials of a drug-eluting insertable actuator for implant feedback or to indicate the type, size, agent or other characteristic of the implant. Other methods of forming a detectable lacrimal implant can be found in commonly-owned De Juan et al., U.S. Patent Application Publication No. US-2009-0099626, entitled “LACRIMAL IMPLANT DETECTION,” filed Sep. 5, 2008, which is herein incorporated by reference in its entirety.

In some examples, the lacrimal implant includes biodegradable elastic materials. These materials can include cross-linked polymers, such as poly (vinyl alcohol). In some examples, the implant body 302 can comprise a silicone/polyurethane co-polymer. Other co-polymers that can be used to form the implant body 302 include, but are not limited to, silicone/urethane, silicone/poly (ethylene glycol) (PEG), and silicone/2hydroxyethyl methacrylate (HEMA). As discussed in commonly-owned Utkhede et al., U.S. Patent Publication No. US-2009-0104243, entitled “DRUG CORES FOR SUSTAINED RELEASE OF THERAPEUTIC AGENTS,” filed Sep. 5, 2008, which is herein incorporated by reference in its entirety, urethane-based polymer and copolymer materials allow for a variety of processing methods and bond well to one another.

In some examples, a lubricious coating disposed on, or impregnated in, an outer surface of the implant body 302 can be used to further aid insertion of the lacrimal implant 300 into the anatomical tissue 352. In one example, the lubricious coating can include a silicone lubricant.

In various examples, the outer surface of the implant body 302 can be formed, or surface treated to be, generally smooth to inhibit bacteria from attaching to the lacrimal implant 300 and incubating. The generally smooth outer surface can also prevent damage to the inner lining of the receiving anatomical tissue, such as a lacrimal punctum 212, 214 (FIG. 2) or associated canaliculus 208, 210 (FIG. 2), during implantation. As further discussed in commonly-owned Rapacki et al., U.S. Patent Publication No. US-2009-0298390, entitled “SURFACE TREATMENT OF IMPLANTS AND RELATED METHODS,” filed Sep. 5, 2008, which is herein incorporated by reference in its entirety, the outer surface of the implant body 302 can be surface treated to be generally smooth via a polishing process. The polishing process can include causing a molded implant body 302 to be impacted with polishing media during an ongoing period of time in which the body 302 is in an enlarged, swelled state. This can smooth one or more surfaces or edges of the implant body 302. In various examples, the polishing media can include at least some granules that are greater than about 3 millimeters in diameter.

In various examples, an antimicrobial coating can be disposed on or impregnated in at least a portion of the outer surface to further prevent bacteria growth on the implant body 302. In some examples, the antimicrobial coating can include an agent selected from the group consisting of 2-bromo-2-nitropropane-1,3-diol, 5-bromo-5-nitro-1,3-dioxane, 7-ethyl bicyclooxazolidine, benzalkonium chloride, benzethonium chloride, benzoic acid, benzyl alcohol, boric acid, bronopol, cetylpyridinium chloride, chlorhexidine digluconate, chloroacetamide, chlorobutanol, chloromethyl isothiazolinone and methyl isothiazoline, dimethoxane, dimethyl oxazolidine, dimethyl hydroxymethylpyrazole, chloroxylenol, dehydroacetic acid, diazolidinyl urea, dichlorobenzyl alcohol, DMDM hydantoin, ethyl alcohol, formaldehyde, glutaraldehyde, hexachlorophene, hexetidine, hexamethylenetramine, imidazolidinyl urea, iodopropynyl butylcarbamate, isothiazolinones, methenammonium chloride, methyldibromo glutaronitrile, MDM hydantoin, minocycline, ortho phenylphenol, p-chloro-m-cresol, parabens (butylparaben, ethylparaben, methylparaben), phenethyl alcohol, phenoxyethanol, piroctane olamine, polyaminopropyl biguanide, polymethoxy bicyclic oxazolidine, polyoxymethylene, polyquaternium-42, potassium benzoate, potassium sorbate, propionic acid, quaternium-15, rifampin, salicylic acid, selenium disulfide, sodium borate, sodium iodate, sodium hydroxymethylglycinate, sodium propionate, sodium pyrithione, sorbic acid, thimerosal, triclosan, triclocarban, undecylenic acid, zinc phenosulfonate, and zinc pyrithione. In some examples, the antimicrobial coating can include a material selected from the group consisting of silver lactate, silver phosphate, silver citrate, silver acetate, silver benzoate, silver chloride, silver iodide, silver iodate, silver nitrate, silver sulfadiazine, silver palmitate, or one or more mixtures thereof. In some examples, the antimicrobial coating can include at least one of an antibiotic or an antiseptic. For instance, the antimicrobial coating can include a temporary anesthetic lasting, on average, between a few hours and a day. In still other examples, the antimicrobial coating can include a drug or other therapeutic agent used to treat an underlying disease, such as a bolus, for immediate effect.

In some examples, the lacrimal implant 300 is included in a kit. The kit may contain one or more lacrimal implants and one or more instructions for using the lacrimal implant to treat an eye disorder or other type of disorder.

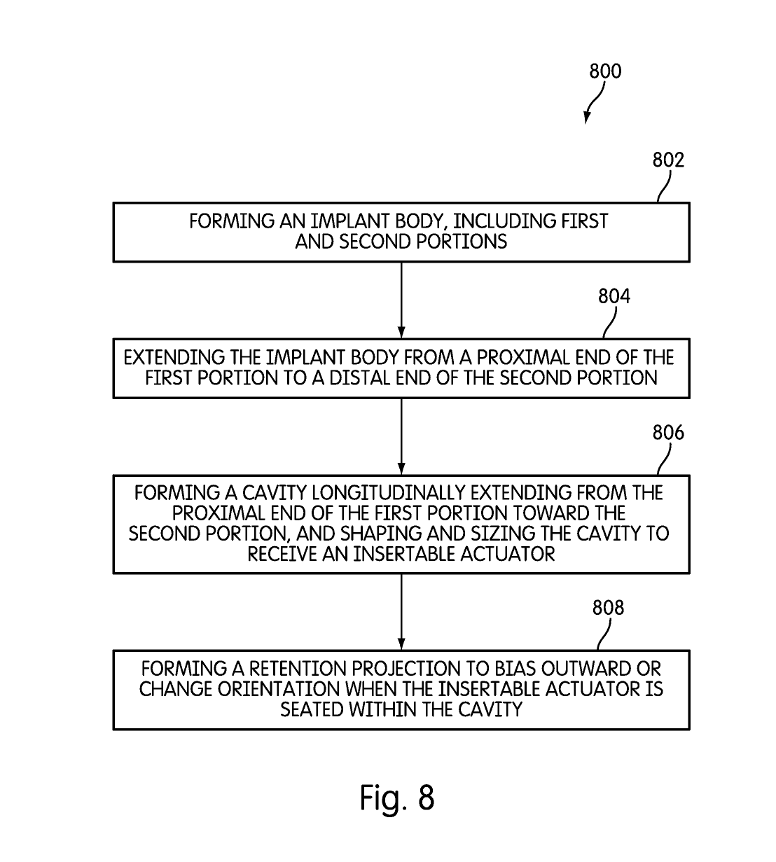

FIG. 8 illustrates an example of a method of manufacturing a lacrimal implant insertable into a lacrimal canaliculus. At block 802, an implant body having first and second portions is formed. At block 804, the implant body is extended from a proximal end of the first portion to a distal end of the second portion.

At block 806, a cavity is formed in the implant body that extends longitudinally from the proximal end of the first portion toward the second portion. The cavity is shaped and sized to receive an insertable actuator.

At block 808, a retention projection is formed within the second portion. The retention projection is configured to bias outward or to change orientation when the insertable actuator is seated within the cavity.

In some examples, the cavity extends into the retention projection of the implant body second portion. In varying examples, the diameter or width of the cavity in the retention projection is less then the diameter or width of the insertable actuator. The insertable actuator thus biases the retention actuator outwardly when the insertable actuator is sufficiently seated in the cavity of the retention projection.

In certain examples, the method includes positioning the insertable actuator at least partially into the cavity. The insertable actuator is not seated in the cavity to the extent necessary to outwardly bias the retention projection. When the lacrimal implant is inserted into the lacrimal canaliculus, the insertable actuator can then be seated to the extent necessary to bias the retention projection outward.

In some examples, the method includes disposing at least one therapeutic agent in the insertable actuator. In this way, the insertable actuator serves the dual purpose of actuating the retention of the lacrimal implant in the lacrimal canaliculus and providing a therapeutic agent (e.g., by sustained release) while the lacrimal implant is implanted. In some examples, the method includes forming a sheath around the insertable actuator to implement agent release.

### Sheath Body Examples:

In various ways, the sheath body can comprise appropriate shapes and materials to control migration of drug or other therapeutic agents from a distinct drug insert or an implant body including integrated drug or other agent. In some examples, the sheath body is configured to be conformable to an implant anatomy, such as an anatomy of a lacrimal punctum or associated canaliculus. In some examples, the sheath body at least partially covers or surrounds the drug insert and can fit snugly against an outer surface of a matrix/agent mixture. In other examples, the sheath body covers or surrounds portions of an implant body including one or more integrated agents. The sheath body can be made from a material that is substantially impermeable to the drug or other therapeutic agent so that the rate of migration of the drug or agent is largely controlled by an exposed surface area of the drug insert or implant body that is not covered by the sheath body. In many examples, migration of the agents through the sheath body can be about one tenth of the migration of the agent through the exposed surface of the drug insert, or less.

Suitable sheath body materials can include, among others, polyimide, polyethylene terephthalate (PET), or parylene. The sheath body can have a thickness, as defined from the sheath surface adjacent the outer matrix/agent mixture surface to an opposing sheath surface away from the outer surface, of about 0.00025 inches to about 0.0015 inches. The total diameter of the sheath that extends across a drug insert can range from about 0.2 millimeters to about 1.2 millimeters. The drug insert can be formed by dip coating the matrix in the sheath body. In some examples, the sheath body can comprise a tube into which the matrix/agent mixture is introduced. The sheath body can also be dip coated around the matrix/agent mixture, for example dip coated around a pre-formed matrix/agent core or implant body.

The sheath body can be provided with one or more additional features such as to facilitate clinical use of the lacrimal implants discussed herein. For example, the sheath can receive a drug insert that is exchangeable in situ, while the implant body remains implanted in the patient, or after its removal. In some examples, the sheath body can be provided with one or more external protrusions that apply force to the sheath body when squeezed, which cause the matrix/agent mixture to be ejected from the sheath body. A replacement drug insert can then be positioned in the sheath body.

### Therapeutic Agent Examples:

A therapeutic agent (or simply “agent”) can comprise, among other things, a drug made from one or any combination of the following or their equivalents, derivatives or analogs, including, anti-glaucoma medications, (e.g. adrenergic agonists, adrenergic antagonists (beta blockers), carbonic anhydrase inhibitors (CAIs, systemic and topical), parasympathomimetics, prostaglandins and hypotensive lipids, and combinations thereof), antimicrobial agent (e.g., antibiotic, antiviral, antiparacytic, antifungal, etc.), a corticosteroid or other anti-inflammatory (e.g., an NSAID or other analgesic and pain management compounds), a decongestant (e.g., vasoconstrictor), an agent that prevents of modifies an allergic response (e.g., an antihistamine, cytokine inhibitor, leucotriene inhibitor, IgE inhibitor, immunomodulator), a mast cell stabilizer, cycloplegic, mydriatic or the like.

Example available agents include, but are not limited to, thrombin inhibitors; antithrombogenic agents; thrombolytic agents; fibrinolytic agents; vasospasm inhibitors; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non steroidal anti-inflammatories (NSAIDs) (such as salicylate, indomethacin, ibuprofen, diclofenac, flurbiprofen, piroxicam indomethacin, ibuprofen, naxopren, piroxicam and nabumetone). Examples of such anti-inflammatory steroids contemplated for use with the present lacrimal implants, include triamcinolone acetonide (generic name) and corticosteroids that include, for example, triamcinolone, dexamethasone, fluocinolone, cortisone, prednisolone, flumetholone, and derivatives thereof); antiallergenics (such as sodium chromoglycate, antazoline, methapyriline, chlorpheniramine, cetrizine, pyrilamine, prophenpyridamine); anti proliferative agents (such as 1,3-cis retinoic acid, 5-fluorouracil, taxol, rapamycin, mitomycin C and cisplatin); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil3; immunological drugs (such as vaccines and immune stimulants); hormonal agents (such as estrogens,—estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotrapin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix; components; ACE inhibitors; free radical scavengers; chelators; antioxidants; anti polymerases; photodynamic therapy agents; gene therapy agents; and other therapeutic agents such as prostaglandins, antiprostaglandins, prostaglandin precursors, including antiglaucoma drugs including beta-blockers such as Timolol, betaxolol, levobunolol, atenolol, and prostaglandin analogues such as bimatoprost, travoprost, latanoprost etc; carbonic anhydrase inhibitors such as acetazolamide, dorzolamide, brinzolamide, methazolamide, dichlorphenamide, diamox; and neuroprotectants such as lubezole, nimodipine and related compounds; and parasympathomimetrics such as pilocarpine, carbachol, physostigmine and the like.

Additional agents that can be used with the present lacrimal implants include, but are not limited to, drugs that have been approved under Section 505 of the United States Federal Food, Drug, and Cosmetic Act or under the Public Health Service Act, some of which can be found at the U.S. Food and Drug Administration (FDA) website http://www.accessdata.fda.gov/scripts/cder/drugsatfda/index. The present lacrimal implants can also be used with drugs listed in the Orange Book, either in paper or in electronic form, which can be found at the FDA Orange Book website (http://www.fda.gov/cder/ob/)), that has or records the same date as, earlier date than, or later date than, the filing date of this patent document. For example, these drugs can include, among others, dorzolamide, olopatadine, travoprost, bimatoprost, cyclosporin, brimonidine, moxifloxacin, tobramycin, brinzolamide, aciclovir timolol maleate, ketorolac tromethamine, prednisolone acetate, sodium hyaluronate, nepafenac, bromfenac,diclofenac, flurbiprofen, suprofenac, binoxan, patanol, dexamethasone/tobramycin combination, moxifloxacin, or acyclovir.

Examples of diseases or disorders that can be treated with above-listed agents include, but are not limited to, glaucoma, pre- and post-surgical ocular treatments, dry eye, anti-eye allergy, anti-infective, post-surgical inflammation or pain, respiration-related disorders, such as allergies, inner ear disorders, such as dizziness or migraines, or other systemic disorders, such as hypertension, cholesterol management, pulmonary disorders or immunological disorders. In some examples, the therapeutic agent can include a lubricant or a surfactant, for example a lubricant to treat dry eye. In other examples, the therapeutic agent can include an absorbent capable of absorbing tear from an eye.

### Drug Core Examples:

A drug core or insert can comprise one or more drugs or other therapeutic agents, and in some examples, one or more matrix materials to provide sustained release of the drug or other agents. Similarly, where greater amounts of agent are desired, substantial portions of an implant body can comprise one or more integrated drugs or other agents and matrix materials configured to provide release of the agents.

The one or more drugs or other therapeutic agents can migrate from an exposed surface of the drug insert to the target tissue based, at least in part, on a solubility of the drugs or agents in the matrix. The rate of migration of the drugs or agents from the exposed surface can also be related to the concentration of drugs or agents dissolved in the matrix. In some examples, the concentration of drugs or agents dissolved in the drug insert can be controlled to provide the desired release rate of the drugs or agents. In addition or in combination, the rate of migration of drugs or agents from the exposed surface can be related to one or more properties of the matrix in which the drugs or agents dissolve, such as the properties of a silicone matrix formulation. In some examples, the drugs or agents included in the drug insert can include liquid, solid, solid gel, solid crystalline, solid amorphous, solid particulate, or dissolved forms. In one such example, liquid Latanoprost droplets or solid Bimatoprost particles are dispersed in a silicone matrix.

The drug core or drug insert can comprise one or more biocompatible materials capable of providing a sustained release of the one or more drugs or agents. Although the drug core is primarily discussed above with respect to an example comprising a matrix including a substantially non-biodegradable silicone matrix with dissolvable inclusions of the drugs or agents located therein, the drug insert can include other structures that provide sustained release of the drugs or agents, for example a biodegradable matrix, a porous drug insert, a liquid drug insert or a solid drug insert. A matrix that includes the drugs or agents can be formed from either biodegradable or non-biodegradable polymers. In some examples, a non-biodegradable drug insert can include silicone, acrylates, polyethylenes, polyurethane, polyurethane, hydrogel, polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, polymer foam, silicone rubber, polyethylene terephthalate, ultra high molecular weight polyethylene, polycarbonate urethane, polyurethane, polyimides, stainless steel, nickel-titanium alloy (e.g., Nitinol), titanium, stainless steel, cobalt-chrome alloy (e.g., ELGILOY® from Elgin Specialty Metals, Elgin, Ill.; CONICHROME®. from Carpenter Metals Corp., Wyomissing, Pa.). In some examples, a biodegradable drug insert can comprise one or more biodegradable polymers, such as protein, hydrogel, polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof. In some examples, the drug core can comprise a hydrogel polymer.

### Lacrimal Implant Inserter Tool Examples

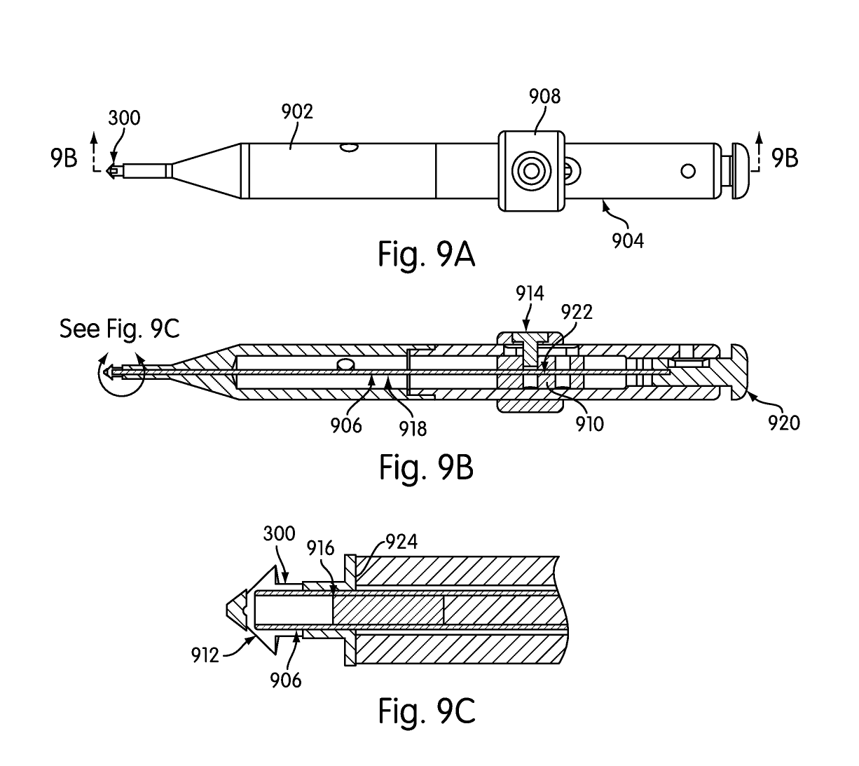

FIGS. 9A and 9B illustrate an example of an insertion tool 900 that can be used to insert the lacrimal implants described herein. The body of the insertion tool 900 includes a front portion 902 and a back portion 904. A lacrimal implant 300 (see, e.g., FIG. 3) is held on the end of the insertion tool 900 by a hollow tube 906, which can be mostly internal to the insertion tool body. A stop 924 at a distal end of the insertion tool body engages a proximal end of the lacrimal implant body. A distal end of the hollow tube 906 is extendable to a position beyond the stop 924, and is sized to be at least partially insertable into the cavity of the lacrimal implant 300. The hollow tube 906 is advanced into the cavity by a tool actuator 908 to hold the lacrimal implant 300 in place. The hollow tube 906 runs through a lumen or barrel 910. In some examples, the hollow tube 906 is secured to the barrel 910 using glue 922 (e.g., Loctite®). The tool actuator 908 may be secured to the barrel 910 using a fastener such as a screw 914.

The distal end of the hollow tube 906 is sized to hold at least a portion of the insertable actuator 916. Insertion of the insertable actuator 916 causes a retention projection of the lacrimal implant 300 to be outwardly biased or change its orientation. In some examples, the insertable actuator 916 is partially pre-loaded into the lacrimal implant 300. An elongate member 918 is slidable within the hollow tube 906 to engage the insertable actuator 916 and to insert the insertable actuator 916 from the hollow tube 906 into the lacrimal implant cavity.

In certain examples, the elongate member includes a wire. In certain examples, the elongate member 918 includes a shaft made of a non-metal material such as plastic. An actuator (e.g., button 920) at the back of the insertion tool is attached to the elongate member 918.

FIG. 9C shows that the button 920 can be used to advance the elongate member 918 in order to advance the insertable actuator 916 into the lacrimal implant 300. Typically the insertable actuator 916 is advanced when the lacrimal implant 300 is inserted into a lacrimal punctum. The insertable actuator 916 outwardly biases or expands a retention projection 912 of the lacrimal implant 300 when the insertable actuator is seated in the cavity of the lacrimal implant 300.

The distal end of the hollow tube 906 is retractable to a position proximal the insertion tool stop 924 to release the lacrimal implant 300. After insertion of the lacrimal implant 300 into the lacrimal punctum, the hollow tube 906 and elongate member 918 are retracted using the tool actuator 908, leaving the lacrimal implant 300 inserted with the retention projection 912 outwardly biased.

FIGS. 9A-9C show the insertion tool in relation to the straight implant of FIG. 3 and FIG. 5. The insertion tool 900 also can be effective to insert lacrimal implants, such as those represented in FIG. 7.

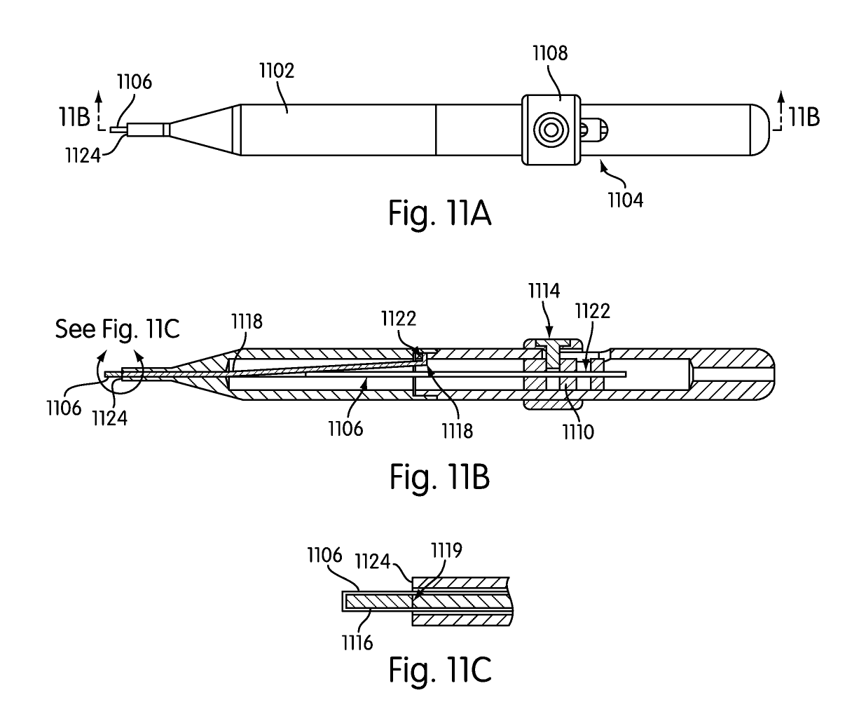

FIGS. 11A and 11B illustrate an example of an insertion tool 1100 that can be used to insert the lacrimal implants described herein. The body of the insertion tool 1100 includes a front portion 1102 and a back portion 1104. A lacrimal implant (not shown) is held on the end of the insertion tool 1100 by a hollow tube 1106, which can be mostly internal to the insertion tool body. A stop 1124 at a distal end of the insertion tool body engages a proximal end of the lacrimal implant body. A distal end of the hollow tube 1106 is extendable to a position beyond the stop 1124, and is sized to be at least partially insertable into the cavity of the lacrimal implant. The hollow tube 1106 is advanced into the cavity by a tool actuator 1108 to hold the lacrimal implant in place. The hollow tube 1106 runs through a lumen or barrel 1110. In some examples, the hollow tube 1106 is secured to the barrel 1110 using glue 1122 (e.g., Loctite®). The tool actuator 1108 may be secured to the barrel 1110 using a fastener such as a screw 1114.

The distal end of the hollow tube 1106 is sized to hold at least a portion of the insertable actuator 1116. Insertion of the insertable actuator 1116 causes a retention projection of the lacrimal implant to be outwardly biased or change its orientation. In some examples, the insertable actuator 1116 is partially pre-loaded into the lacrimal implant. An elongate member 1118 is secured to the front portion 1102 of the insertion tool 1100 using glue 1122 (e.g., Loctite®). In certain examples, the elongate member 1118 includes a wire. In certain examples, the elongate member 1118 includes a shaft made of a non-metal material such as plastic. A distal end 1119 of the elongated member 1118 is positioned to be flush with the stop 1124, and may engage the insertable actuator 1116 when the insertable actuator 1116 is located within the hollow tube 1106, as illustrated in FIG. 11C.

Typically, the insertable actuator 1116 is discharged when the lacrimal implant is inserted into a lacrimal punctum. The insertable actuator 1116 outwardly biases or expands a retention projection of the lacrimal implant when the insertable actuator is seated in the cavity of the lacrimal implant. After insertion of the lacrimal implant into the lacrimal punctum, the hollow tube 1106 is retracted using the tool actuator 1108, leaving the lacrimal implant inserted with the retention projection outwardly biased. When the distal end of hollow tube 1106 is retracted to a position proximal the insertion tool stop 1124, the distal end 1119 of the elongated member 1118 prevents the insertable actuator 1116 from entering the front portion 1102 of the body of the insertion tool 1100 and allows the insertable actuator 1116 to be discharged from the hollow tube 1106 and into the lacrimal implant cavity as the lacrimal implant is released from the insertion tool 1100.

Among other things, lacrimal implants and related methods providing secure retention within a lacrimal punctum and canaliculus of an eye are discussed. The lacrimal implants can comprise an implant body configured for at least partial insertion through the lacrimal punctum and into the associated canaliculus. The implant body can include first and second portions, and can extend from a proximal end of the first portion defining a longitudinal proximal axis to a distal end of the second portion defining a longitudinal distal axis. The implant body can be configured such that, when implanted using an integral dilator, an at least 45 degree angled intersection exists between the proximal axis and the distal axis. In this way, at least a portion of the implant body can be biased against at least a portion of the lacrimal canaliculus located at or more distal to a canalicular curvature, thereby retaining an implanted position of the lacrimal implant using anatomical structures.

In various examples, the lacrimal implant can further comprise a drug insert disposed in at least one of the first portion or the second portion of the implant body to provide a sustained release of a drug or other therapeutic agent to an eye, nasal passage, or inner ear system, for instance. The drug insert can include a distinct drug core disposed within an implant body cavity or can include a mixture of drug or other agent particles throughout one or more implant body portions, or both.

Advantageously, in some examples, the present lacrimal implants can successfully block the flow of tears or provide sustained delivery of a drug or other therapeutic agent to an eye, nasal passage, or inner ear for varying periods of time, such as from days to months to years. In addition, by optionally including first and second implant body cavities or drug releasing implant body portions, a dual drug or other agent releasing profile can be possible. For instance, two separate drugs can be released from two different implant locations. Further, the canalicular retaining configuration of the present implant body can reduce over-stretching of the lacrimal punctum and canaliculus and inadvertent fall out of implants. It is believe the present lacrimal implants can, but need not, be implemented so-as-to provide a one-size-fits-all regime. The expandable nature of the present lacrimal can allow for easier implantation, as some of the retention features of the implant can be activated post-implantation.

The present lacrimal implant may also be better retained within a punctum and canaliculus of a patient due to the combination of, for example, a cap-like projection at a proximal end of a first implant portion, a heel-like retainment projection at a proximal end of a second implant portion, or one or more intermediate or distally located projections on the first or second implant portions. As further discussed above, the cap-like projection may inhibit the implant wholly from migrating below the punctum and into the lacrimal canaliculus. The intermediate, distal and heel-like projections may help hold the implant in place until a caregiver physician chooses to remove it.

The insertable actuators (e.g., drug cores), lacrimal implants, and methods of manufacturing the same, as referred to in this patent document, can take any one of a number of different designs, configurations, or arrangements beyond those listed above, such as are described in the following commonly-owned patent documents, each of which is incorporated herein by reference in its entirety: U.S. Patent Publication No. US-2007-0269487, entitled “DRUG DELIVERY METHODS, STRUCTURES, AND COMPOSITIONS FOR NASOLACRIMAL SYSTEM,” filed Apr. 2, 2007; U.S. Patent Application Ser. No. 60/970,755, entitled “PROSTAGLANDIN ANALOGUES FOR IMPLANT DEVICES AND METHODS,” filed Sep. 7, 2007; U.S. Patent Application Ser. No. 61/252,057, entitled “LACRIMAL IMPLANTS AND RELATED METHODS,” filed on Oct. 15, 2009; U.S. Patent Publication No. U.S. Patent Publication No. US-2009-01042748, entitled “LACRIMAL IMPLANTS AND RELATED METHODS,” filed Sep. 5, 2008; U.S. Patent Publication No. US-2010-0034870, entitled “COMPOSITE LACRIMAL INSERT AND RELATED METHODS,” filed Apr. 29, 2009; U.S. Patent Publication No. US-2010-0274224, entitled “LACRIMAL IMPLANT BODY INCLUDING COMFORTING AGENT,” filed Jul. 8, 2009; and U.S. Patent Publication No. US-2005-0232972, entitled “DRUG DELIVERY VIA PUNCTAL PLUG,” filed Apr. 15, 2004.

The above Detailed Description includes references to the accompanying drawings, which form a part of the Detailed Description. The drawings show, by way of illustration, specific embodiments in which the invention can be practiced. These embodiments are also referred to herein as “examples.” All publications, patents, and other patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the term “about” is used to refer to an amount that is approximately, nearly, almost, or in the vicinity of being equal to a stated amount.

In this document, the term “proximal” refers to a location relatively closer to the cornea of an eye, and the term “distal” refers to a location relatively further from the cornea and inserted deeper into a lacrimal canaliculus.

In this document, the term “hydrogel” is used to refer to an absorbing or otherwise retaining material (e.g., adsorbing material), such as super-absorbent polymers, hydrocolloids, and water-absorbent hydrophilic polymers, for example. Examples of hydrogels for use with the present lacrimal implants include, among others, aliphatic thermoplastic polyurethanes (TPU), such as hydrophilic, aliphatic, and polyether-based thermoplastic polyurethanes. Suitable thermoplastic polyurethanes include those commercially available from the Lubrizol Corporation of Cleveland, Ohio under the trade name, Tecophilic. In certain applications, hydrogels commercially available under the trade names “Tecophilic TG-500” (or simply “TG-500”) and “Tecophilic TG-2000” (or simply “TG-2000”) can be utilized. The term “hydrogel” can refer to super-absorbent polymer particles in a “dry” state, such as when the hydrogel is not expanded and contains less to no water weight. The term “hydrogel” can also be used to refer to super-absorbent polymer particles in a hydrated or expanded state, more specifically, hydrogels that have absorbed at least their weight in water, such as several hundred times their weight in water (e.g., TG-500, which can absorb about 500 times its weight in water and TG-2000, which can absorb about 2000 times its weight in water). As the hydrogel material absorbs fluid, its size can increase (e.g., swell) and its shape can change to bias against, or cause a surrounding material to bias against, at least a portion of a lacrimal ampulla or lacrimal canalicular wall.

In the appended claims, the terms “including” and “in which” are used as the plain-English equivalents of the respective terms “comprising” and “wherein.” Also, in the following claims, the terms “including” and “comprising” are open-ended, that is, a system, assembly, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above Detailed Description is intended to be illustrative, and not restrictive. For example, the above-described examples (or one or more features thereof) can be used in combination with each other. As an example, one or more dimensions from the various implant embodiments shown or described may be grouped together to form an implant embodiment capable of providing a desired drug concentration. Other embodiments can be used, such as by one of ordinary skill in the art upon reviewing the above description. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

1. A lacrimal implant insertable at least partially into a lacrimal punctum, the lacrimal implant comprising:
an implant body, including first and second portions, the implant body extending from a proximal end of the first portion to a distal end of the second portion;
the second portion including a retention projection; and
a cavity longitudinally extending from the proximal end of the first portion toward the second portion, wherein the cavity is shaped and sized to receive an insertable actuator, and wherein the retention projection is configured to bias outward or change orientation when the insertable actuator is seated within the cavity.

2. The lacrimal implant of claim 1, wherein the lacrimal implant includes a cut or void extending from at least a portion of the cavity to at least partially through the retention projection and substantially transverse to the longitudinal extension of the cavity.

3. The lacrimal implant of claim 2, wherein the retention projection includes at least a first portion and a second portion, wherein the first portion of the retention projection is biased in a first outward direction and the second portion of the retention projection is biased in a second outward direction when the insertable actuator is seated in the cavity.

4. The lacrimal implant of claim 3, wherein the cut or void extends through the retention projection to define the first portion and the second portion of the retention projection.

5. The lacrimal implant of claim 2, wherein the cavity extends at least partially through the cut or void to receive the insertable actuator at least partially into the cut or void.

6. The lacrimal implant of claim 1, wherein the proximal end of the first portion of the implant body defines a longitudinal proximal axis and the distal end of the second portion of the implant body defines a longitudinal distal axis, and
wherein a non-linear angled intersection exists between the proximal axis and the distal axis for biasing at least a portion of the implant body against at least a portion of the lacrimal canaliculus located at, or located more distal, to a canalicular curvature.

7. The lacrimal implant of claim 6, wherein a proximal end of the second portion is biased toward a lacrimal canaliculus ampulla when the insertable actuator is seated in the cavity.

8. The lacrimal implant of claim 6, wherein a sidewall of the cavity is biased inward and the retention projection is biased inward toward the first portion when the insertable actuator is not seated within the cavity.

9. The lacrimal implant of claim 1, wherein the cavity longitudinally extends into, but not through, the second portion, such that a distal end of the cavity resides in the retention projection.

10. The lacrimal implant of claim 1, wherein a width of the cavity at the distal end is less than a width of the cavity at the proximal end.

11. The lacrimal implant of claim 1, including an insertable actuator configured to expand when placed in the cavity.

12. The lacrimal implant of claim 1, wherein the proximal end of the first portion includes a graspable projection laterally protruding around at least a portion of its circumference.

13. The lacrimal implant of claim 12, wherein a perimeter of the graspable projection is numerically about equal to a perimeter of the retainment projection proximal end.

14. The lacrimal implant of claim 1, further comprising the insertable actuator, wherein the insertable actuator includes one or more therapeutic agents.

15. The lacrimal implant of claim 1, further comprising one or more therapeutic agents integrated with the implant body.

16. The lacrimal implant of claim 14, wherein the one or more therapeutic agents include a sustained release ocular agent.

17. The lacrimal implant of claim 14, wherein the one or more therapeutics agents are deliverable, on a sustained release basis, to tissue of a nasolacrimal system.

18. The lacrimal implant of claim 14, wherein the one or more therapeutics agents are deliverable, on a sustained release basis, to tear film fluid of an eye.

19. The lacrimal implant of claim 1, wherein the implant body comprises an inert, non-expanding material.

20. A kit comprising the lacrimal implant of claim 1, and an instruction for using the lacrimal implant to treat an eye disorder.

21. The kit of claim 18, including an insertable actuator.

22. A method of manufacturing a lacrimal implant insertable into a lacrimal canaliculus, the method comprising:
forming an implant body, including first and second portions;
extending the implant body from a proximal end of the first portion to a distal end of the second portion;
forming a cavity longitudinally extending from the proximal end of the first portion toward the second portion, and shaping and sizing the cavity to receive an insertable actuator; and
forming a retention projection in the second portion to bias outward or change orientation when the insertable actuator is seated within the cavity.

23. The method of claim 20, including positioning the insertable actuator at least partially into the cavity.

24. The method of claim 20, including disposing at least one therapeutic agent in the insertable actuator.

25. A lacrimal implant insertable at least partially into a lacrimal punctum, the lacrimal implant comprising:
an implant body, including first and second portions, the implant body extending from a proximal end of the first portion to a distal end of the second portion;
the second portion including a retention projection; and
an insertable actuator positionable within a cavity of the implant body, the cavity longitudinally extending from the proximal end of the first portion toward the second portion, wherein the retention projection is configured to bias outward or change orientation when the insertable actuator is seated within the cavity.

26. The lacrimal implant of claim 25, wherein the insertable actuator includes one or more therapeutic agents.

27. The lacrimal implant of claim 25, wherein the insertable actuator includes a sheath body surrounding one or more surfaces of the insertable actuator.

28. The lacrimal implant of claim 25, wherein the lacrimal implant includes a cut or void extending from at least a portion of the cavity to at least partially through the retention projection and substantially transverse to the longitudinal extension of the cavity.

29. The lacrimal implant of claim 25, wherein the proximal end of the first portion of the implant body defines a longitudinal proximal axis and the distal end of the second portion of the implant body defines a longitudinal distal axis, and
wherein a non-linear angled intersection exists between the proximal axis and the distal axis for biasing at least a portion of the implant body against at least a portion of the lacrimal canaliculus located at, or located more distal, to a canalicular curvature.

30. The lacrimal implant of claim 29, wherein a proximal end of the second portion is biased toward a lacrimal canaliculus ampulla when the insertable actuator is seated in the cavity.

31. The lacrimal implant of claim 25, wherein the insertable actuator is configured to be removable from the cavity after implant.

32. A kit comprising:
the lacrimal implant of claim 25; and
a lacrimal implant insertion tool including:
a stop at a distal end of a body of the insertion tool to engage a proximal end of the implant body;
a hollow tube at least partially internal to the insertion tool body and having a distal end extendable beyond the stop, wherein the distal end of the hollow tube is sized to be at least partially insertable into the lacrimal implant cavity and sized to hold at least a portion of the insertable actuator, and wherein the distal end of the hollow tube is retractable to a position proximal the stop to release the lacrimal implant; and
an elongate member slidable within the hollow tube to urge the insertable actuator from the hollow tube into the lacrimal implant cavity.

33. The kit of claim 32, wherein the stop limits the penetration depth of the lacrimal implant into the lacrimal punctum.

34. The kit of claim 32, wherein the elongate member includes a wire.

35. The kit of claim 32, including an instruction for using the lacrimal implant to treat an eye disorder, and wherein the insertable actuator includes one or more therapeutic agents.
